(19) United States
(12) Patent Application Publication
(10) Pub. No.: US 2007/0006397 A1
(43) Pub. Date: Jan. 11, 2007

(54) Colorants for keratin fibers

(76) Inventors: Jurgen Schmenger, Weiterstadt (DE); Jolanthe Kujawa, Darmstadt (DE)

Correspondence Address: THE PROCTER & GAMBLE COMPANY;INTELLECTUAL PROPERTY DIVISION, WINTON HILL BUSINESS CENTER - BOX 161, 6110 CENTER HILL AVENUE, CINCINNATI, OH, 45224, US

(21) Appl. No.: 11/441,896

(22) Filed: May 26, 2006

(30) Foreign Application Priority Data
May 27, 2005 (DE) 202005008307.0

(51) Int. Cl. A61K 8/00

(52) U.S. Cl. 8/405

(57) ABSTRACT

The present patent application relates to an agent for the coloring of keratin fibers based on oxidative dye precursors and/or direct-penetrating dyes, that contains a combination of at least one cationic hydroxyethyl cellulose and at least one acrylate copolymer in a suitable cosmetic carrier as well as a multicomponent-kit for the coloring of keratin fibers.

### FIELD OF THE INVENTION

The present invention relates to agents for coloring keratin fibers, in particular human hair, containing direct-penetrating and/or oxidative dyes, as well as a combination of cationic hydroxyethyl celluloses and an acrylate copolymer.

### BACKGROUND OF THE INVENTION

Coloring preparations generally take the form of aqueous—preferably thickened—solutions or emulsions and in addition to dyes contain for example fatty alcohols and/or other oil components, emulsifiers and surfactants, as well as alcohols if necessary. Oxidative dyes as a rule consist of two components, (i) the dye vehicle that contains the dye, and (ii) the oxidizing agent preparation, which shortly before use are mixed together and then applied to the hair that is to be colored. A conditioning step is desirable after rinsing out the oxidative dye. A more or less well-pronounced conditioning effect is obtained whenever a conditioning raw material is added, but by no means can this replace the use of a subsequently applied conditioning agent.

Depending on the viscosity and mixture ratio of the two components, a higher or lower viscosity will be obtained upon mixing. Thus, good adhesion of the colorant is achieved especially well through the colorant having a higher viscosity. Moreover, hairdressers often require high viscosity in the materials they use in their work, for example in certain highlighting and lowlighting techniques or foiling techniques as well as for being able to perform spot treatments with a color brush or an accent brush.

There is thus a significant need for an inexpensive material for the thickening of dye carriers, which will ensure good miscibility of the dye carrier with the oxidizing agent and will provide colorants that have good adhesive properties and coloring properties, and that moreover will exhibit improved conditioning properties in comparison with agents of the prior art.

The use of cationic hydroxyethyl celluloses in hair dyes is reasonably well known. With regard to their viscosity and adhesion to the hair, however, the conditioning properties and coloring properties of such agents are not satisfactory in all respects. The viscosity of an oxidative dye is raised only marginally by the cellulose derivatives used in the prior art and such a preparation will possess only an unsatisfactory conditioning effect.

### SUMMARY OF THE INVENTION

It has now been discovered, surprisingly, that the aforementioned disadvantages of the cellulose derivatives that have been used up to now are very effectively removed by using a combination of cationic cellulose derivatives and acrylate copolymers, and that it is possible to achieve both an excellent conditioning effect and a very good increase in viscosity.

### DETAILED DESCRIPTION OF THE INVENTION

The object of the present invention is thus an agent for the coloring of keratin fibers such as hair, especially human hair, based on oxidative dye precursors and/or direct-penetrating dyes, wherein said agent contains a combination of at least one cationic hydroxyethyl cellulose and at least one acrylate copolymer in a suitable cosmetic carrier.

The cationic hydroxyethyl celluloses and the acrylate copolymers are contained in the colorant according to the present invention preferably in a quantity of from about 0.01 to 20 percent by weight, in particular from 0.1 to approximately 5 percent by weight (calculated on the basis of the active substance).

Cationic hydroxyethyl celluloses (INCI Polyquaternium 10) are distributed for example under the trade names Celquat SC-240C (National Starch Co.), Rita Polyquata 400 (Rita Co.), or Ucare Polymer JR types (Amerchol Co.), among others.

Acrylate copolymers (INCI acrylate copolymer) are distributed for example under the trade names Aculyn 33 (Rohm&Haas Co.), Avalure AC 120 (Noveon Co.), Balance 0/55 (National Starch Co.), Capigel 98 (Seppic Co.), and others. The use of methacrylic acid/ethyl acrylate copolymers, as distributed by the Seppic Co. under the trade name Capigel 98, is especially preferred.

The colorant according to the present invention preferably contains oxidative dye precursors, through which the coloring is produced by the action of oxidizing agents, such as for example hydrogen peroxide, or in the presence of atmospheric oxygen.

The following are some examples of developer substances and coupler substances, as well as self-coupling compounds, that are suitable oxidative dye precursors:
(i) Developer substances: 1,4-diaminobenzene (p-phenylendiamine), 1,4-diamino-2-methylbenzene (p-toluenediamine), 1,4-diamino-2,6-dimethylbenzene, 1,4-diamino-3,5-diethylbenzene, 1,4-diamino-2,5-dimethylbenzene, 1,4-diamino-2,3-dimethylbenzene, 2-chloro-1,4-diaminobenzene, 1,4-diamino-2-(thiophen-2-yl)benzene, 1,4-diamino-2-(thiophen-3-yl)benzene, 1,4-diamino-2-(pyridin-3-yl)benzene, 2,5-diaminobiphenyl, 1,4-diamino-2-methoxymethylbenzene, 1,4-diamino-2-aminomethylbenzene, 1,4-diamino-2-hydroxymethylbenzene, 1,4-diamino-2-(2-hydroxyethoxy)benzene, 2-(2-(acetylamino)ethoxy)-1,4-diaminobenzene, 4-phenylaminoaniline, 4-dimethylaminoaniline, 4-diethylaminoaniline, 4-dipropylaminoaniline, 4-[ethyl(2-hydroxyethyl)amino]aniline, 4-[di-(2-hydroxyethyl)amino]aniline, 4-[di-(2-hydroxyethyl)amino]-2-methylaniline, 4-[(2-methoxyethyl)amino]aniline, 4-[(3-hydroxypropyl)amino]aniline, 4-[(2,3-dihydroxypropyl)amino]aniline, 1,4-diamino-2-(2-hydroxyethyl)benzene, 1,4-diamino-2-(1-methylethyl)benzene, 1,3-bis-[(4-aminophenyl)(2-hydroxyethyl)amino]-2-propanol, 1,4-bis-[(4-aminophenyl)amino]butane, 1,8-bis-(2,5-diaminophenoxy)-3,6-dioxaoctane, 4-aminophenol, 4-amino-3-methylphenol, 4-amino-3-(hydroxymethyl)phenol, 4-amino-3-fluorophenol, 4-methylaminophenol, 4-amino-2-(aminomethyl)phenol, 4-amino-2-(hydroxymethyl)phenol, 4-amino-2-fluorophenol, 4-amino-2-[(2-hydroxyethyl)amino]methylphenol, 4-amino-2-methylphenol, 4-amino-2-(methoxymethyl)phenol, 4-amino-2-(2-hydroxyethyl)phenol, 5-aminosalicylic acid, 2,5-diaminopyridine, 2,4,5,6-tetraaminopyrimidine, 2,5,6-triamino-4-(1H)-pyrimidone, 4,5-diamino-1-(2-hydroxyethyl)-1H-pyrazole, 4,5-diamino-1-(1-methylethyl)-1H-pyrazole, 4,5-diamino-1-[(4-methylphenyl)methyl]-1H-pyrazole, 1-[(4-chlorophenyl)methyl]-4,5-diamino-1H-pyrazole, 4,5-diamino-1-methyl-1H-pyrazole, 2-aminophenol, 2-amino-6-methylphenol, 2-amino-5-methylphenol, alone or in mixtures with each other.
(ii) Coupler substances: N-(3-dimethylaminophenyl)urea, 2,6-diaminopyridine, 2-amino-4-[(2-hydroxyethyl)amino]anisole, 2,4-diamino-1-fluoro-5-methylbenzene, 2,4-diamino-1-methoxy-5-methylbenzene, 2,4-diamino-1-ethoxy-5-methylbenzene, 2,4-diamino-1-(2-hydroxyethoxy)-5-methylbenzene, 2,4-di-[(2-hydroxyethyl)amino]-1,5-dimethoxybenzene, 2,3-diamino-6-methoxypyridine, 3-amino-6-methoxy-2-(methylamino)pyridine, 2,6-diamino-3,5-dimethoxypyridine, 3,5-diamino-2,6-dimethoxypyridine, 1,3-diaminobenzene, 2,4-diamino-1-(2-hydroxyethoxy)benzene, 1,3-diamino-4-(2,3-dihydroxypropoxy)benzene, 2,4-diamino-1,5-di-(2-hydroxyethoxy)benzene, 1-(2-aminoethoxy)-2,4-diaminobenzene, 2-amino-1-(2-hydroxyethoxy)-4-methylaminobenzene, 2,4-diaminophenoxyacetic acid, 3-[di-(2-hydroxyethyl)amino]aniline, 4-amino-2-di-[(2-hydroxyethyl)amino]-1-ethoxybenzene, 5-methyl-2-(1-methylethyl)phenol, 3-[(2-hydroxyethyl)amino]aniline, 3-[(2-aminoethyl)amino]aniline, 1,3-di-(2,4-diaminophenoxy)propane, di-(2,4-diaminophenoxy)methane, 1,3-diamino-2,4-dimethoxybenzene, 2,6-bis-(2-hydroxyethyl)aminotoluene, 4-hydroxyindole, 3-dimethylaminophenol, 3-diethylaminophenol, 5-amino-2-methylphenol, 5-amino-4-fluoro-2-methylphenol, 5-amino-4-methoxy-2-methylphenol, 5-amino-4-ethoxy-2-methylphenol, 3-amino-2,4-dichlorophenol, 5-amino-2,4-dichlorophenol, 3-amino-2-methylphenol, 3-amino-2-chloro-6-methylphenol, 3-aminophenol, 3-amino-2,6-dimethylphenol, 2-methyl-5-(β-hydroxyethylamino)phenol, 5-amino-2-ethylphenol, 2-[(3-hydroxyphenyl)amino]-acetamide, 5-[(2-hydroxyethyl)amino]-4-methoxy-2-methylphenol, 5-[(2-hydroxyethyl)amino]-2-methylphenol,3-[(2-hydroxyethyl)amino]phenol, 3-[(2-methoxyethyl)amino]phenol, 5-amino-2-ethylphenol, 5-amino-2-methoxyphenol, 2-(4-amino-2-hydroxyphenoxy)ethanol, 5-[(3-hydroxypropyl)amino]-2-methylphenol, 3-[(2,3-dihydroxypropyl)amino]-2-methylphenol, 3-[(2-hydroxyethyl)amino]-2-methylphenol, 2-amino-3-hydroxypyridine, 5-amino-4-chloro-2-methylphenol, 1-naphthol, 2-methyl-1-naphthol, 1,5-dihydroxynaphthalene, 1,7-dihydroxynaphthalene, 2,3-dihydroxynaphthalene, 2,7-dihydroxynaphthalene, 2-methyl-1-naphthol acetate, 1,3-dihydroxybenzene, 1-chloro-2,4-dihydroxybenzene, 2-chloro-1,3-dihydroxybenzene, 1,2-dichloro-3,5-dihydroxy-4-methylbenzene, 1,5-dichloro-2,4-dihydroxybenzene, 1,3-dihydroxy-2-methylbenzene, 3,4-methylenedioxyphenol, 3,4-methylenedioxyaniline, 5-[(2-hydroxyethyl)amino]-1,3-benzodioxole, 6-bromo-1-hydroxy-3,4-methylenedioxybenzene, 3,4-diaminobenzoic acid, 3,4-dihydro-6-hydroxy-1,4(2H)-benzoxazine, 6-amino-3,4-dihydro-1,4(2H)-benzoxazine, 3-methyl-1-phenyl-5-pyrazolone, 5,6-dihydroxyindole, 5,6-dihydroxyindoline, 5-hydroxyindole, 6-hydroxyindole, 7-hydroxyindole, 2,3-indoline dione, alone or in mixtures with each other.
(iii) Self-coupling compounds: 2-amino-5-methylphenol, 2-amino-6-methylphenol, 2-amino-5-ethoxyphenol, or 2-propylamino-5-aminopyridine.

The total quantity of the oxidative dye precursors contained in the agent according to the present invention is approximately 0 to 12 percent by weight, especially approximately 0.2 to 6 percent by weight.

To obtain specific color shades, moreover, additional conventional natural and/or synthetic direct-penetrating dyes can be contained in the colorant, including for example plant pigments such as henna or indigo, triphenylmethane dyes, aromatic nitro dyes, azo dyes, quinone dyes, cationic or anionic dyes.

Examples of suitable synthetic dyes include: 1,4-bis-[(2-hydroxyethyl)amino]-2-nitrobenzene, 1-(2-hydroxyethyl)amino-2-nitro-4-[di-(2-hydroxyethyl)amino]benzene (HC Blue No. 2), 1-amino-3-methyl-4-[(2-hydroxyethyl)amino]-6-nitrobenzene (HC Violet No. 1), 4-[ethyl-(2-hydroxyethyl)amino]-1-[(2-hydroxyethyl)amino]-2-nitrobenzene hydrochloride (HC Blue No. 12), 4-[di-(2-hydroxyethyl)amino]-1-[(2-methoxyethyl)amino]-2-nitrobenzene (HC Blue No.11), 1-[(2,3-dihydroxypropyl)amino]-4-[methyl-(2-hydroxyethyl)amino]-2-nitrobenzene (HC Blue No. 10), 1-[(2,3-dihydroxypropyl)amino]-4-[ethyl-(2-hydroxyethyl)amino]-2-nitrobenzene hydrochloride (HC Blue No. 9), 1-(3-hydroxypropylamino)-4-[di-(2-hydroxyethyl)amino]-2-nitrobenzene (HC Violet No. 2), 1-methylamino-4-[methyl-(2,3-dihydroxypropyl)amino]-2-nitrobenzene (HC Blue No. 6), 2-((4-amino-2-nitrophenyl)amino)-5-dimethylaminobenzoic acid (HC Blue No. 13), 1-amino-4-[(2-hydroxyethyl)amino]-2-nitrobenzene (HC Red No. 7), 2-amino-4,6-dinitrophenol, 4-amino-2-nitrodiphenylamine (HC Red No. 1), 1-amino-4-[di-(2-hydroxyethyl)amino]-2-nitrobenzene hydrochloride (HC Red No. 13), 1-amino-5-chloro-4-[(2-hydroxyethyl)amino]-2-nitrobenzene, 4-amino-1-[(2-hydroxyethyl)amino]-2-nitrobenzene (HC Red No. 3), 4-amino-3-nitrophenol, 4-[(2-hydroxyethyl)amino]-3-nitrophenol, 1-[(2-aminoethyl)amino]-4-(2-hydroxyethoxy)-2-nitrobenzene (HC Orange No. 2), 4-(2,3-dihydroxypropoxy)-1-[(2-hydroxyethyl)amino]-2-nitrobenzene (HC Orange No. 3), 1-amino-5-chloro-4-[(2,3-dihydroxypropyl)amino]-2-nitrobenzene (HC Red No. 10), 5-chloro-1,4-[di-(2,3-dihydroxypropyl)amino]-2-nitrobenzene (HC Red No.11), 2-[(2-hydroxyethyl)amino]-4,6-dinitrophenol, 4-ethylamino-3-nitrobenzoic acid, 2-[(4-amino-2-nitrophenyl)amino]benzoic acid, 2-chloro-6-ethylamino-4-nitrophenol, 2-amino-6-chloro-4-nitrophenol, 4-[(3-hydroxypropyl)amino]-3-nitrophenol, 2,5-diamino-6-nitropyridine, 1,2,3,4-tetrahydro-6-nitroquinoxaline, 7-amino-3,4-dihydro-6-nitro-2H-1,4-benzoxazine (HC Red No. 14), 1-amino-2-[(2-hydroxyethyl)amino]-5-nitrobenzene (HC Yellow No. 5), 1-(2-hydroxyethoxy)-2-[(2-hydroxyethyl)amino]-5-nitrobenzene (HC Yellow No. 4), 1-[(2-hydroxyethyl)amino]-2-nitrobenzene (HC Yellow No. 2), 2-[(2-hydroxy-ethyl)amino]-1-methoxy-5-nitrobenzene, 2-amino-3-nitrophenol, 1-(2-hydroxyethoxy)-3-methylamino-4-nitrobenzene, 2,3-(dihydroxypropoxy)-3-methylamino-4-nitrobenzene, 2-[(2-hydroxyethyl)amino]-5-nitrophenol (HC Yellow No. 11), 3-[(2-aminoethyl)amino]-1-methoxy-4-nitrobenzene hydrochloride (HC Yellow No. 9), 1-[(2-ureidoethyl)amino]-4-nitrobenzene, 4-[(2,3-dihydroxypropyl)amino]-3-nitro-1-trifluoromethylbenzene (HC Yellow No. 6), 1-chloro-2,4-bis-[(2-hydroxyethyl)amino]-5-nitrobenzene (HC Yellow No. 10), 4-[(2-hydroxyethyl)amino]-3-nitro-1-methylbenzene, 1-chloro-4-[(2-hydroxyethyl)amino]-3-nitrobenzene (HC Yellow No. 12), 4-[(2-hydroxyethyl)amino]-3-nitro-1-trifluoromethylbenzene (HC Yellow No. 13), 4-[(2-hydroxyethyl)amino]-3-nitrobenzonitrile (HC Yellow No. 14), 4-[(2-hydroxyethyl)amino]-3-nitrobenzamide (HC Yellow No. 15), 1,4-di-[(2,3-dihydroxypropyl)amino]-9,10-anthraquinone, 1-[(2-hydroxyethyl)amino]-4-methylamino-9,10-anthraquinone (CI61505, Disperse Blue No. 3), 2-[(2-aminoethyl)amino]-9,10-anthraquinone (HC Orange No. 5), 1-hydroxy-4-[(4-methyl-2-sulfophenyl)amino]-9,10-anthraquinone, 1-[(3-aminopropyl)amino]-4-methylamino-9,10-anthraquinone (HC Blue No. 8), 1-[(3-aminopropyl)amino]-9,10-anthraquinone (HC Red No. 8), 1,4-diamino-2-methoxy-9,10-anthraquinone (CI62015, Disperse Red No. 11, Solvent Violet No. 26), 1,4-dihydroxy-5,8-bis-[(2-hydroxyethyl)amino]-9,10-anthraquinone (CI62500, Disperse Blue No. 7, Solvent Blue No. 69), 9-(dimethylamino)benzo[a]phenoxazin-7-ium chloride (CI51175; Basic Blue No. 6), 1-((4-amino-3,5-dimethylphenyl)-(2,6-dichlorophenyl)methylene)-3,5-dimethyl-4-imino-2,5-cyclohexadiene with phosphoric acid (1:1) (Basic Blue 77), di-[4-(diethylamino)phenyl] [4-(ethylamino)naphthyl]carbenium chloride (CI42595; Basic Blue No. 7), 3,7-di-(dimethylamino)phenothiazin-5-ium chloride (CI52015; Basic Blue No. 9), di-[4-(dimethylamino)phenyl][4-(phenylamino)naphthyl]-carbenium chloride (CI44045; Basic Blue No. 26), 2-[(4-(ethyl-(2-hydroxyethyl)amino)-phenyl)azo]-6-methoxy-3-methylbenzothiazolium methyl sulfate (CI11154; Basic Blue No. 41), 8-amino-2-bromo-5-hydroxy-4-imino-6-[(3-(trimethylammonio)phenyl)amino]-1(4H)naphthalenone chloride (CI56059; Basic Blue No. 99), bis-[4-(dimethylamino)-phenyl]-[4-(methylamino)phenyl]carbenium chloride (CI42535; Basic Violet No. 1), tris-[4-(dimethylamino)phenyl]carbenium chloride (CI42555; Basic Violet No. 3), 2-[3,6-(diethylamino)dibenzopyranium-9-yl]benzoic acid chloride (CI45170; Basic Violet No. 10), di-(4-aminophenyl)-(4-amino-3-methylphenyl)carbenium chloride (CI42510; Basic Violet No. 14), 1,3-bis-[(2,4-diamino-5-methylphenyl)azo]-3-methylbenzene (CI21010; Basic Brown No. 4), 1-[(4-aminophenyl)azo]-7-(trimethylammonio)-2-naphthol chloride (CI12250; Basic Brown No. 16), 1-[(4-amino-2-nitrophenyl)azo]-7-(trimethylammonio)-2-naphthol chloride (Basic Brown No. 17), 1-[(4-amino-3-nitrophenyl)azo]-7-(trimethylammonio)-2-naphthol chloride (CI1 2251; Basic Brown No. 17), 3,7-diamino-2,8-dimethyl-5-phenylphenazinium chloride (CI50240; Basic Red No. 2), 1,4-dimethyl-5-[(4-(dimethylamino)phenyl)azo]-1,2,4-triazolium chloride (CI 1055; Basic Red No. 22), 2-hydroxy-1-[(2-methoxyphenyl)azo]-7-(trimethylammonio)naphthalene chloride (CI12245; Basic Red No. 76), 2-[2-((2,4-dimethoxyphenyl)amino)ethenyl] -1,3,3-trimethyl-3H-indol-1-ium chloride (CI48055; Basic Yellow No. 11), 3-methyl-1-phenyl-4-[(3-(trimethylammonio)phenyl)azo]pyrazol-5-one chloride (CI12719; Basic Yellow No. 57), bis-[4-(diethylamino)phenyl]phenylcarbenium bisulfate (1:1) (CI42040; Basic Green No. 1), 1-[di-(2-hydroxyethyl)amino]-3-methyl-4-[(4-nitrophenyl)azo]benzene (CI11210, Disperse Red No. 17), 4-[(4-aminophenyl)azo]-1-[di-(2-hydroxyethyl)amino]-3-methylbenzene (HC Yellow No. 7), 2,6-diamino-3-[(pyridin-3-yl)azo]pyridine, 6-hydroxy-5-[(4-sulfophenyl)azo]-2-naphthalenesulfonic acid disodium salt (CI15985; Food Yellow No. 3; FD&C Yellow No. 6), 2,4-dinitro-1-naphthol-7-sulfonic acid disodium salt (CI10316; Acid Yellow No. 1; Food Yellow No. 1), 2-(indan-1,3-dion-2-yl)quinoline-x,x-sulfonic acid (mixture of mono- and disulfonic acid) (CI47005;D&C Yellow No. 10; Food Yellow No. 13; Acid Yellow No. 3), 5-hydroxy-1-(4-sulfophenyl)-4-[(4-sulfophenyl)azo]pyrazole-3-carboxylic acid trisodium salt (CI19140; Food Yellow No. 4; Acid Yellow No. 23), 9-(2-carboxyphenyl)-6-hydroxy-3H-xanthen-3-one (CI45350; Acid Yellow No. 73; D&C Yellow No. 8), 5-[(2,4-dinitrophenyl)amino]-2-phenylaminobenzenesulfonic acid sodium salt (CI 0385; Acid Orange No. 3), 4-[(2,4-dihydroxyphenyl)azo]benzenesulfonic acid monosodium salt (CI14270; Acid Orange No. 6), 4-[(2-hydroxynaphth-1-yl)azo]benzenesulfonic acid sodium salt (CI15510; Acid Orange No. 7), 4-[(2,4-dihydroxy-3-[(2,4-dimethylphenyl)azo]phenyl)azo]-benzenesulfonic acid sodium salt (CI20170; Acid Orange No. 24), 4-hydroxy-3-[(4-sulfonaphth-1-yl)azo]-1-naphthalenesulfonic acid disodium salt (CI14720; Acid Red No. 14), 6-hydroxy-5-[(4-sulfonaphth-1-yl)azo]-2,4-naphthalenedisulfonic acid trisodium salt (CI16255; Ponceau 4R; Acid Red No. 18), 3-hydroxy-4-[(4-sulfonaphth-1-yl)azo]-2,7-naphthalenedisulfonic acid trisodium salt (CI16185; Acid Red No. 27), 8-amino-1-hydroxy-2-(phenylazo)-3,6-naphthalenedisulfonic acid disodium salt (CI17200; Acid Red No. 33), 5-(acetylamino)-4-hydroxy-3-[(2-methylphenyl)azo]-2,7-naphthalenedisulfonic acid disodium salt (CI18065; Acid Red No. 35), 2-(3-hydroxy-2,4,5,7-tetraiododibenzopyran-6-on-9-yl)benzoic acid disodium salt (CI45430;Acid Red No. 51), N-[6-(diethylamino)-9-(2,4-disulfophenyl)-3H-xanthen-3-ylidene]-N-ethylethanammonium hydroxide, inner salt, sodium salt (CI45 100; Acid Red No. 52), 8-[(4-(phenylazo)phenyl)azo]-7-naphthol-1,3-disulfonic acid disodium salt (CI27290; Acid Red No. 73), 2′,4′,5′,7′-tetrabromo-3′,6′-dihydroxyspiro[isobenzofuran-1(3H),9′-[9H]xanthen]-3-one disodium salt (CI45380; Acid Red No. 87), 2′,4′,5′,7′-tetrabromo-4,5,6,7-tetrachloro-3′,6′-dihydroxyspiro[isobenzofuran-1 (3H),9′[9H]xanthen]-3-one disodium salt (CI45410; Acid Red No. 92), 3′,6′-dihydroxy-4′,5′-diiodospiro[isobenzofuran-1(3H),9′(9H)-xanthen]-3-one disodium salt (CI45425; Acid Red No. 95), (2-sulfophenyl)-di-[4-(ethyl((4-sulfophenyl)methyl)amino)phenyl]-carbenium disodium salt, betaine (CI42090; Acid Blue No. 9; FD&C Blue No. 1), 1,4-bis-[(2-sulfo-4-methylphenyl)amino]-9,10-anthraquinone disodium salt (CI 61570; Acid Green No. 25), bis-[4-(dimethylamino)phenyl]-(3,7-disulfo-2-hydroxynaphth-1-yl)carbenium inner salt, monosodium salt (CI44090; Food Green No. 4; Acid Green No. 50), bis-[4-(diethylamino)phenyl](2,4-disulfophenyl)carbenium inner salt, sodium salt (2:1) (CI42045; Food Blue No. 3; Acid Blue No. 1), bis-[4-(diethylamino)phenyl](5-hydroxy-2,4-disulfophenyl)carbenium inner salt, calcium salt (2:1) (CI42051; Acid Blue No. 3), 1-amino-4-(cyclohexylamino)-9,10-anthraquinone-2-sulfonic acid sodium salt (CI62045; Acid Blue No. 62),2-(1,3-dihydro-3-oxo-5-sulfo-2H-indol-2-ylidene)-2,3-dihydro-3-oxo-1H-indole-5-sulfonic acid disodium salt (CI73015; Acid Blue No. 74), 9-(2-carboxyphenyl)-3-[(2-methylphenyl)amino]-6-[(2-methyl-4-sulfophenyl)amino]xanthylium inner salt, monosodium salt (CI45190; Acid Violet No. 9), 1-hydroxy-4-[(4-methyl-2-sulfophenyl)amino]-9,10-anthraquinone sodium salt (CI60730; D&C Violet No. 2; Acid Violet No. 43), bis-[3-nitro-4-[(4-phenylamino)-3-sulfophenylamino]phenyl]sulfone (CI 10410; Acid Brown No. 13), 5-amino-4-hydroxy-6-[(4-nitrophenyl)azo]-3-(phenylazo)-2,7-naphthalenedisulfonic acid disodium salt (CI20470; Acid Black No. 1), 3-hydroxy-4-[(2-hydroxynaphth-1-yl)azo]-7-nitro-1-naphthalenesulfonic acid chromium complex (3:2) (Cl1 5711; Acid Black No. 52), 3-[(2,4-dimethyl-5-sulfophenyl)azo]-4-hydroxy-1-naphthalenesulfonic acid disodium salt (CI14700; Food Red No. 1; FD&C Red No. 4), 4-(acetylamino)-5-hydroxy-6-[(7-sulfo-4-[(4-sulfophenyl)azo]naphth-1-yl)azo]-1,7-naphthalenedisulfonic acid tetrasodium salt (CI28440; Food Black No. 1) and 3-hydroxy-4-(3-methyl-5-oxo-1-phenyl-4,5-dihydro-1H-pyrazol-4-ylazo)naphthalene-1-sulfonic acid sodium salt, chromium complex (Acid Red No. 195), alone or in combination with each other.

The total quantity of direct-penetrating dye in the agent according to the present invention is approximately 0 to 7 percent by weight, preferably from about 0.2 to 4 percent by weight. The dyes can also be present in a separate component and can be added to the agent according to the present invention immediately before use. In this case, the agent according to the present invention does not contain dyes.

Further dyes that are conventional and known for hair coloring that can be contained in the colorant according to the present invention, among others, are described in E. Sagarin, “Cosmetics, Science and Technology,” Interscience Publishers Inc., New York (1957), p. 503 ff. as well as H. in Janistyn, “Handbuch der Kosmetika und Riechstoffe,” Vol. 3 (1973), p. 388 ff. and K. Schrader, “Grundlagen und Rezepturen der Kosmetika,” 2nd ed., (1989), pp. 782-815; reference to the contents of these texts is hereby expressly incorporated.

Although oxidative dyes are preferred, it is of course also possible for the colorant according to the present invention to be present in the form of a nonoxidative colorant based on the aforementioned direct-penetrating dyes.

Furthermore, the agent according to the present invention can contain antioxidants such as ascorbic acid, mercaptoacetic acid or sodium sulfite, as well as chelating agents for heavy metals, for example, ethylenediaminotetraacetate or nitriloacetic acid, in a quantity of up to approximately 0.5 percent by weight. The color vehicle according to the present invention can contain perfume oils in an amount of up to about 0.1 percent by weight. Of course, the above-described hair dye can, if necessary, contain further additives that are customary for hair dyes, one example of which is preservatives; another example of which is solvents such as water, lower aliphatic alcohols, for example aliphatic alcohols with from 1 to 4 carbon atoms such as ethanol, propanol and isopropanol, or glycols such as glycerin and 1,2-propylene glycol; or wetting agents or emulsifiers from the classes of anionic, cationic, amphoteric or non-ionogenic surface-active substances; furthermore, softening agents, vaselines, silicone oils, paraffin oil and fatty acids, as well as conditioners such as cationic resins, lanolin derivatives, cholesterol, vitamins, pantothenic acid and betaine, may be contained. The abovementioned components are used in the usual amounts for such applications; for example, the wetting agents and emulsifiers are used in concentrations of from 0.1 to 30 percent by weight and the conditioning agents in a concentration of from 0.1 to 5.0 percent by weight. Especially advantageous in this regard is the addition of nonionic and/or anionic surfactants or emulsifiers, such as for example fatty alcohol sulfates, especially lauryl sulfate and sodium cocoyl sulfate, oxyethylated fatty alcohol sulfates, especially sodium lauryl ether sulfates with from 2 to 4 ethylene oxide units per molecule, oxyethylated fatty acid esters, oxyethylated nonylphenols, oxyethylated fatty alcohols, alkylbenzene sulfonates or fatty acid alkanolamides, in a total quantity of from about 0.1 to 30 percent by weight, preferably 0.2 to 15 percent by weight.

The pH value of the colorant according to the present invention for nonoxidative colorants based on direct-penetrating dyes will be in the range of from about 5 to 10, preferably from 6 to 9, while for oxidative colorants based on oxidative dye precursors the pH value will be in the range of from about 6 to 12, preferably from 9 to 11, the pH value of the ready-to-use oxidative hair colorant (i.e., the mixture of the hair dye according to the present invention with the oxidizing agent) being from about 5.5 to 10, preferably from 6 to 9.

Depending on the composition and desired pH value of the colorant, the adjustment of the pH value is preferably carried out with ammonia or organic amines, such as for example glucamines, aminomethylpropanol, monoethanolamine or triethanolamine, inorganic bases, for example sodium hydroxide, potassium hydroxide, sodium carbonate or calcium hydroxide, or respectively with organic or inorganic acids, such as for example lactic acid, citric acid, acetic acid or phosphoric acid.

The agent according to the present invention is preferably formulated as an aqueous or aqueous alcoholic preparation, for example as a thickened solution, as an emulsion, as a creme or as a gel. For use in oxidative coloring, the above-described colorant is mixed immediately before use with an oxidizing agent, and a quantity of the ready-to-use preparation that is sufficient for coloring is applied to the fibers, generally from about 2.1 oz-7.1 oz (60 g to 200 g). If the agent according to the present invention is formulated without dyes, the dyes are present as a separate component that can be mixed with an oxidizing agent and with the agent according to the present invention immediately before use. The agent according to the present invention can also be formulated as an oxidizing agent, and a colorant having dyes, or dyes that are present separately, can be added. The agent according to the present invention can also be present separately, as what is known as a thickening additive, and in addition the colorant and the oxidizing agent can be present in already-mixed form.

If the dyes/dye precursors are present as separate components, they are preferably confectioned in the form of a powder, a granulate, or a pellet (for example in the form of the dye pellets described in DE 103 47 242 A1).

Provided that the colorant according to the present invention contains no oxidative dye precursors, or contains oxidative dye precursors that are easily oxidizable with atmospheric oxygen, it can be applied directly to the keratin fibers without being mixed with an oxidizing agent beforehand.

Possible oxidizing agents for developing the coloring primarily include hydrogen peroxide or its addition compounds with urea, melanin, or sodium borate, in the form of a 1 to 12 percent, preferably 1.5 to 6 percent, aqueous solution. The mixture ratio of colorant to oxidizing agent depends on the concentration of the oxidizing agent and will generally be from about 5:1 to 1:2, preferably 1: 1, the content of the oxidizing agent in the ready-to-use preparation being preferably from about 0.5 to 8 percent by weight, especially from 1 to 4 percent by weight.

The ready-to-use colorant is allowed to act on the keratin fibers (for example human hair) at a temperature of from 15° C. to 50° C. for a period of from about 10 to 45 minutes, preferably from about 15 to 30 minutes; the fibers are then rinsed with water and dried. If necessary, the fibers can be washed with a shampoo after this rinsing, and possibly post-rinsed with a weak organic acid, such as for example tartaric acid. The keratin fibers are subsequently dried.

If necessary, the viscosity of the oxidizing colorant according to the present invention can also easily be further increased after the mixing with the oxidizing agent, by adding the thickener combination according to the present invention of at least one cationic hydroxyethyl cellulose and at least one acrylate copolymer; in this way, simpler and more economical base formulations are possible. It is also possible to add the thickener combination according to the present invention, made up of at least one cationic hydroxyethyl cellulose and at least one acrylate copolymer, to the colorant immediately before use (before, after, or during the mixing with the oxidizing agent), and to set the desired viscosity in this way.

The colorant according to the present invention fulfills the stated requirements in an outstanding manner with respect to the adhesive properties, the conditioning properties, the application behavior and the adjustment of the viscosity, and is noticeably easier to apply than comparable agents. Furthermore, the colorants according to the present invention possess a uniform consistency and an attractive cosmetic appearance. Of particular note is the very good viscosity and the outstanding stability of the agent according to the present invention, as well as its excellent adhesion to hair. Furthermore, the application of the agent according to the present invention enables a variation of the weight ratio of colorant to oxidizing agent over a broad range (for example from 1:1 to 1:3) without any noticeable adverse effects on the viscosity or on the adhesion properties of the ready-to-use oxidative dye.

The following Examples should serve to illustrate the subject matter of the invention in more detail, without the invention being limited to these examples.

### EXAMPLES

### Example 1

Creme-type oxidative hair colorant
Component (A): Base creme without dyes

| | | |
|---|---|---|
| 7.0000 | g | cetyl stearyl alcohol |
| 3.0000 | g | glycol distearate |
| 3.0000 | g | polyethylene glycol(25)cetylstearylether |
| 9.0000 | g | coconut acid monoethanolamide |
| 3.0000 | g | polyethylene glycol(7)glycerylmonococoate |
| 4.0000 | g | lauryl ether sulfate |
| 0.5000 | g | methacrylic acid/ethyl acrylate |
| | | copolymer (Capigel ® 98) |
| 0.3000 | g | cationic hydroxyethylcellulose |
| | | (Polyquaternium 10) |
| 0.1000 | g | ethylenediamine acetic acid |
| 0.2000 | g | ascorbic acid |
| 0.4000 | g | sodium sulfite |
| 0.2500 | g | perfume |
| 9.5000 | g | ammonia, 25% aqueous solution |
| balance to 100.0000 | g | water, completely desalinated |

Component (B): Dyes in powder or granulate form

| | |
|---|---|
| 6.810 g | 4-aminophenol |
| 2.500 g | 1-naphthol |
| 0.068 g | resorcinol |
| 0.017 g | 2-amino-6-chloro-4-nitrophenol |

50 g of the existing base creme (A) is mixed immediately before use with 10 g of the existing dye formula (B) and 50 g of a 6 percent aqueous hydrogen peroxide solution. A homogeneous, cosmetically attractive, optimally thickened coloring preparation is obtained. The mixture so obtained is then applied to medium blonde natural hair. After an action period of 30 minutes at 40° C., the hair is rinsed with water and dried. The hair obtains a reddish-brown, radiant color.

### Example 2

Oxidative hair colorant, liquid

| | | |
|---|---|---|
| 0.5000 | g | methacrylic acid/ethyl acrylate copolymer |
| | | (Capigel ® 98) |
| 0.3000 | g | cationic hydroxyethyl cellulose |
| | | (Polyquaternium 10) |
| 5.0000 | g | 2-octyl-1-dodecanol |
| 15.0000 | g | oleic acid |
| 10.0000 | g | sodium lauryl alcohol diglycol ether |
| | | sulfate (28% aqueous solution) |
| 1.3620 | g | 4-aminophenol |
| 0.5000 | g | 1-naphthol |
| 0.0136 | g | resorcinol |
| 0.0034 | g | 2-amino-6-chloro-4-nitrophenol |
| 12.0000 | g | ammonia, 25% aqueous solution |
| 1.0000 | g | ethylenediaminetetraacetic acid disodium salt |
| 1.0000 | g | ascorbic acid |
| 15.0000 | g | isopropanol |
| balance to 100.0000 | g | water |

50 g of the abovementioned hair colorant is mixed immediately before use with 50 g of a 6% aqueous hydrogen peroxide solution. A homogeneous, cosmetically attractive, optimally thickened coloring preparation is obtained. The mixture so obtained is then applied to blonde natural hair. After an action period of 30 minutes at 40° C., the hair is rinsed with water and dried. The hair obtains a radiant, copper-red color.

### Example 3

Gel-type oxidative hair colorant for brilliant coloring
Component (A): Liquid color vehicle

| | | |
|---|---|---|
| 0.70 | g | methacrylic acid/ethyl acrylate copolymer |
| | | (Capigel ® 98) |
| 0.25 | g | cationic hydroxyethylcellulose |
| | | (Polyquaternium 10) |
| 10.00 | g | lauryl alcohol |
| 6.00 | g | nonylphenol, oxethylated with 4 mole ethylene |
| | | oxide |
| 6.00 | g | oleic acid |
| 0.50 | g | p-phenylenediamine |
| 0.07 | g | resorcinol |
| 5.00 | g | sodium lauryl alcohol diglycol ether sulfate, |
| | | 28% aqueous solution |
| 1.00 | g | ethylenediaminetetraacetic acid-disodium salt |
| 18.00 | g | ammonia, 25% aqueous solution |
| 8.00 | g | ethanol |
| balance to 100.00 | g | water |

Component (B): Hydrogen peroxide emulsion

| | | |
|---|---|---|
| 10.0 | g | cetyl stearyl alcohol |
| 1.5 | g | cholesterol |
| 4.0 | g | sodium lauryl alcohol diglycol ether sulfate, 28% |
| | | aqueous solution |
| 35.0 | g | hydrogen peroxide, 35% aqueous solution |
| 0.3 | g | perfume |
| balance to 100.0 | g | water |

Before use, 40 g of the liquid color vehicle (A) is mixed with 80 g of the hydrogen peroxide emulsion (B), corresponding to a mixture ratio of (A):(B) of 1:2, and 120 g of this mixture is applied to gray human hair. After an action period of 20 minutes at room temperature the hair is rinsed with water and dried. The hair treated in this manner is uniformly colored light brown from the hair roots to the hair tips. The agent according to the present invention is easily applied and does not run from the hair.

### Example 4

Oxidative hair colorant, creme-type

| | | |
|---|---|---|
| 0.40 | g | methacrylic acid/ethyl acrylate copolymer |
| | | (Capigel ® 98) |
| 0.40 | g | cationic hydroxyethylcellulose |
| | | (Polyquaternium 10) |
| 3.00 | g | oleyl alcohol |
| 15.00 | g | cetyl alcohol |
| 3.50 | g | sodium lauryl alcohol diglycol ether sulfate, 28% |
| | | aqueous solution |
| 3.00 | g | monoethanolamine |
| 1.30 | g | 1-methyl-2,5-diaminobenzene |
| 1.00 | g | beeswax |
| 0.65 | g | resorcinol |
| 0.50 | g | keratin hydrolysate |
| 0.50 | g | silk protein hydrolysate |
| 0.50 | g | 2-amino-6-chloro-4-nitrophenol |
| 0.30 | g | ascorbic acid |
| balance to 100.00 | g | water |

50 g of the abovementioned hair colorant is mixed immediately before use with 50 g of a 12% aqueous hydrogen peroxide solution. The mixture so obtained is then applied to blonde natural hair. After an action period of 30 minutes at 40° C., the hair is rinsed with water and dried. A uniform, robust brown tint is obtained.

### Example 5

Non-oxidative hair tint

| | | |
|---|---|---|
| 2.50 | g | methacrylic acid/ethyl acrylate copolymer |
| | | (Capigel ® 98) |
| 0.25 | g | cationic hydroxyethylcellulose |
| | | (Polyquaternium 10) |
| 6.00 | g | lauryl alcohol |
| 5.00 | g | sodium lauryl sulfate |
| 3.00 | g | cetyl stearyl alcohol and sodium lauryl sulfate |
| | | (Lanette ® W) |
| 1.50 | g | 2-amino-6-chloro-4-nitrophenol |
| 1.00 | g | monoethanolamine |
| 1.00 | g | beeswax |
| 0.50 | g | keratin hydrolysate |
| 0.30 | g | silk protein hydrolysate |
| 0.20 | g | glycine |
| balance to 100.00 | g | water |

A slightly gel-like coloring mass is obtained that can be applied easily and uniformly, and also adheres well to the hair, as a result of its outstanding viscosity properties. After an action period of 20 minutes at 20° C., the hair is rinsed with lukewarm water, set in a style and dried. The hair treated in this way exhibits a uniform, robust, radiant golden-orange color.

### Example 6

Oxidative hair colorant, creme-type
Component (A): Color vehicle

| | | |
|---|---|---|
| 1.5 | g | acrylate copolymer (acrylate copolymer) |
| 0.3 | g | cationic hydroxyethylcellulose |
| | | (Polyquaternium 10) |
| 8.0 | g | 2-octyl-1-dodecanol |
| 3.0 | g | sodium lauryl alcohol diglycol ether sulfate, 28% |
| | | aqueous solution |
| 2.8 | g | 2,5-diamino toluene sulfate |
| 1.0 | g | resorcinol |
| 0.4 | g | m-aminophenol |
| 0.2 | g | 2-amino-4-(2′-hydroxyethylamino)anisole sulfate |
| 0.3 | g | ascorbic acid |
| 0.1 | g | ethylenediaminetetraacetic acid |
| 12.2 | g | ammonia, 25% aqueous solution |
| 2.0 | g | ethanol |
| balance to 100.0 | g | water |

Component (B): Hydrogen peroxide - emulsion

| | | |
|---|---|---|
| 10.0 | g | cetyl stearyl alcohol |
| 1.5 | g | cholesterol |
| 4.0 | g | sodium lauryl alcohol diglycol ether sulfate, 28% |
| | | aqueous solution |
| 17.0 | g | hydrogen peroxide, 35% aqueous solution |
| 0.3 | g | perfume |
| balance to 100.0 | g | water |

Before use, 40 g of the liquid color vehicle (A) is mixed with 80 g of the hydrogen peroxide emulsion (B), corresponding to a mixture ratio of (A):(B) 1:2, and 120 g of this mixture is applied to gray human hair. After an action period of 20 minutes at room temperature the hair is rinsed with water and dried. The hair treated in this manner has obtained a uniform, dark brown tint. The agent according to the present invention adheres very well to the hair without running off.

### Example 7

oxidative hair colorant
Component (A): Color vehicle

| | | |
|---|---|---|
| 0.75 | g | methacrylic acid/ethyl acrylate copolymer |
| | | (Capigel ® 98) |
| 0.3 | g | cationic hydroxyethylcellulose |
| | | (Polyquaternium 10) |
| 8.00 | g | 2-octyl-1-dodecanol |
| 9.0 | g | cetyl stearyl alcohol |
| 3.00 | g | sodium lauryl alcohol diglycol ether sulfate, 28% |
| | | aqueous solution |
| 0.30 | g | ascorbic acid |
| 0.10 | g | ethylenediaminetetraacetic acid |
| 12.20 | g | ammonia, 25% aqueous solution |
| 2.00 | g | ethanol |
| balance to 100.00 | g | water |

The dyes are added to the color vehicle in powder or granulate/pellet form. Examples of granulate base tints are listed in the following Table 1.

TABLE 1

| | BASE TINTS: | | | | | | | | |
|---|---|---|---|---|---|---|---|---|---|
| | (Dye amount in | | | | | | | | |
| | grams) | | | | | | | | |
| | yellow | golden | orange | red | purple | pink | blue | brown | green |
| 2,5-diamino- | | | | | | | | | 4.95 |
| toluene sulfate | | | | | | | | | |
| 2,4-diamino- | | | | | | | 3.86 | | |
| phen-oxyethanol | | | | | | | | | |
| *HCl | | | | | | | | | |
| p-phenylene- | | | | | | | | 2.40 | |
| diamine | | | | | | | | | |
| p-aminophenol | | | 1.50 | | | | | | |
| resorcinol | | | | | | | | 1.80 | |
| m-aminophenol | | | | | | | | 0.64 | |
| 4-amino-2- | | | 2.05 | 1.12 | 1.17 | | | | |
| hydroxytoluene | | | | | | | | | |
| 1-naphthol | | | | | | 1.73 | | | |
| 6-amino-m- | 1.50 | | | | | | | | |
| cresol | | | | | | | | | |
| hydroxyethyl- | | | | | | | | | 4.90 |
| 3,4-methylene- | | | | | | | | | |
| dioxy-aniline | | | | | | | | | |
| *HCl | | | | | | | | | |
| N,N-bis-(2- | | | | | 2.80 | | 4.71 | | |
| hydroxyethyl)- | | | | | | | | | |
| p-phenylene- | | | | | | | | | |
| diamine sulfate | | | | | | | | | |
| 1-hydroxyethyl- | | | | 2.18 | | 2.88 | | | |
| 4,5-diamino- | | | | | | | | | |
| pyrazole sulfate | | | | | | | | | |
| 2-amino-6- | | 2.00 | | | | | | | |
| chloro-4- | | | | | | | | | |
| nitrophenol | | | | | | | | | |
| N-(2-hydroxy- | | | | | | | | | 3.00 |
| ethyl)-2-nitro-4- | | | | | | | | | |
| trifluoromethyl- | | | | | | | | | |
| aniline | | | | | | | | | |

The base tints can optionally be mixed with each other to achieve a wide variety of shades.

Component (B): Hydrogen peroxide emulsion

| | | |
|---|---|---|
| 10.0 | g | cetyl stearyl alcohol |
| 1.5 | g | cholesterol |
| 4.0 | g | sodium lauryl alcohol diglycol ether sulfate, |
| | | 28% aqueous solution |
| 35.0 | g | hydrogen peroxide, 35% aqueous solution |
| 0.3 | g | perfume |
| balance to 100.0 | g | water |

Before use, 40 g of the creme-type color vehicle (A) is mixed with 80 g of the hydrogen peroxide emulsion (B), corresponding to a mixture ratio of (A):(B) 1:2, to which mixtures of granular base tints are added; the entire preparation is stirred and then 120 g of this mixture is applied to gray human hair. After an action period of 20 minutes at room temperature the hair is rinsed with water and dried. The hair treated in this manner has obtained a tint depending on the mixture of base tints. This agent according to the present invention adheres very well to the hair without running off.

### Example 8

### Oxidative Hair Colorant, Acrylate Copolymer in the Peroxide Component

Component (A): Color vehicle

| | | |
|---|---|---|
| 8.0 | g | 2-octyl-1-dodecanol |
| 3.0 | g | sodium lauryl alcohol diglycol ether sulfate, |
| | | 28% aqueous solution |
| 2.8 | g | 2,5-diamino toluene sulfate |
| 1.0 | g | resorcinol |
| 0.4 | g | m-aminophenol |
| 0.2 | g | 2-amino-4-(2′-hydroxyethylamino)anisole sulfate |
| 0.3 | g | ascorbic acid |
| 0.1 | g | ethylenediaminetetraacetic acid |
| 12.2 | g | ammonia, 25% aqueous solution |
| 0.3 | g | cationic hydroxyethylcellulose |
| | | (Polyquaternium 10) |
| 2.0 | g | ethanol |
| balance to 100.0 | g | water |

Component (B): Hydrogen peroxide emulsion

| | | |
|---|---|---|
| 1.2 | g | methacrylic acid/ethyl acrylate copolymer |
| | | (Capigel ® 98) |
| 10.0 | g | cetyl stearyl alcohol |
| 1.5 | g | cholesterol |
| 4.0 | g | sodium lauryl alcohol diglycol ether sulfate, |
| | | 28% aqueous solution |
| 25.7 | g | hydrogen peroxide, 35% aqueous solution |
| 0.3 | g | perfume |
| balance to 100.0 | g | water |

Before use, 40 g of the liquid color vehicle (A) is mixed with 80 g of the hydrogen peroxide emulsion (B), corresponding to a mixture ratio of (A):(B) 1:2, and 120 g of this mixture is applied to gray human hair. After an action period of 20 minutes at room temperature the hair is rinsed with water and dried. The hair treated in this manner has obtained a uniform, dark brown tint. This agent according to the present invention adheres very well to the hair without running off.

### Example 9

### Addition as Subsequent Color Thickener

Component (A): Color thickener with cellulose ether

| | | |
|---|---|---|
| 10.0 | g | methacrylic acid/ethyl acrylate copolymer |
| | | (Capigel ® 98) |
| 3.0 | g | cationic hydroxyethylcellulose |
| | | (Polyquaternium 10) |
| 0.3 | g | perfume |
| 10.0 | g | ethanol |
| balance to 100.0 | g | water |

Component (B): Liquid color vehicle

| | | |
|---|---|---|
| 10.00 | g | lauryl alcohol |
| 6.00 | g | nonylphenol, oxethylated with 4 mole ethylene |
| | | oxide |
| 6.00 | g | oleic acid |
| 0.50 | g | p-phenylenediamine |
| 0.07 | g | resorcinol |
| 5.00 | g | sodium lauryl alcohol diglycol ether sulfate, |
| | | 28% aqueous solution |
| 1.00 | g | ethylenediaminetetraacetic acid-disodium salt |
| 18.00 | g | ammonia, 25% aqueous solution |
| 8.00 | g | ethanol |
| balance to 100.00 | g | water |

Component (C): Hydrogen peroxide emulsion

| | | |
|---|---|---|
| 10.0 | g | cetylstearyl alcohol |
| 1.5 | g | cholesterol |
| 4.0 | g | sodium lauryl alcohol diglycol ether sulfate, |
| | | 28% aqueous solution |
| 35.0 | g | hydrogen peroxide, 35% aqueous solution |
| 0.3 | g | perfume |
| balance to 100.0 | g | water |

Before use, 40 g of the liquid color vehicle (A) is mixed with 40 g of the hydrogen peroxide emulsion (B), corresponding to a mixture ratio of (A):(B) 1:1, and the color thickener is added to this to achieve the desired viscosity (from about 1 to 3 g), and the mixture obtained is applied to gray human hair. After an action period of 20 minutes at room temperature the hair is rinsed with water and is then dried. The hair treated in this manner has obtained a uniform, dark brown tint. This agent according to the present invention adheres very well to the hair without running off.

Unless indicated otherwise, all percentages given in the present application represent weight percents.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An agent for the coloring of keratin fibers based on oxidative dye precursors and/or direct-penetrating dyes, wherein said agent comprises a combination of at least one cationic hydroxyethyl cellulose and at least one acrylate copolymer in a suitable cosmetic carrier.

2. An agent according to claim 1, wherein the acrylate copolymer is a methacrylic acid/ethyl acrylate copolymer.

3. An agent according to claim 1, wherein the cationic hydroxyethyl cellulose and the acrylate copolymer are each present in a quantity of from 0.01 to 20 percent by weight.

4. An agent for the thickening of agents for coloring keratin fibers, comprising a combination of at least one cationic hydroxyethyl cellulose and at least one acrylate copolymer.

5. An agent according to claim 4, wherein the cationic hydroxyethyl cellulose and the acrylate copolymer are each present in a quantity of from 0.01 to 20 percent by weight.

6. An agent according to claim 1, wherein said agent is a hair colorant.

7. An agent according to claim 1, wherein said agent is mixed with an oxidizing agent prior to use.

8. A multicomponent kit for the coloring of keratin fibers, wherein said kit comprises (i) a colorant comprising oxidative dye precursors and/or direct-penetrating dyes, as well as a combination of at least one cationic hydroxyethyl cellulose and at least one acrylate copolymer, and (ii) an oxidizing agent preparation.

9. A multicomponent kit for the coloring of keratin fibers, wherein said kit comprises (i) a colorant containing oxidative dye precursors and/or direct-penetrating dyes and (ii) a thickening agent comprising a combination of at least one cationic hydroxyethyl cellulose and at least one acrylate copolymer.

10. A multicomponent kit for coloring keratin fibers, whrein said multicomponent kit comprises (i) a powdered or granular/pellet-type preparation comprising a dye which is slected from the group consisting of oxidative dye precursors, direct-penetrating dyes and mixtures thereof, and (ii) a dye-free color vehicle comprising a combination of at least one cationic hydroxyethyl cellulose and at least one acrylate copolymer.

11. A multicomponent kit according to claim 9, further comprising (iii) an oxidizing agent preparation.

12. A multicomponent kit according to claim 10, further comprising (iii) an oxidizing agent preparation.
